(19) United States
(12) Patent Application Publication
(10) Pub. No.: US 2005/0154411 A1
(43) Pub. Date: Jul. 14, 2005

(54) Method and apparatus for trephinating body vessels and hollow organ walls

(76) Inventors: Eugene M. Breznock, Winters, CA (US); Jay A. Lenker, Laguna Beach, CA (US)

Correspondence Address: JAY A. LENKER, 408 Panorama Drive, Laguna Beach, CA, 92651, US

(21) Appl. No.: 11/075,268

(22) Filed: Mar. 8, 2005

Related U.S. Application Data
(63) Continuation-in-part of application No. 09/938,428, filed on Aug. 23, 2001, now Pat. No. 6,863,677.

(51) Int. Cl. A61B 17/32

(52) U.S. Cl. 606/184

(57) ABSTRACT

A system is disclosed for creating a hole in a body vessel or hollow organ. Such holes are useful in surgically preparing the hollow organ or body vessel for connection with another hollow organ, body vessel or prosthetic conduit. For example, an assist device is generally connected to the left ventricle through a ventriculotomy created at the apex of the left ventricle. This ventriculotomy is most easily created with a punch or trephine. Control over such a procedure must be precise so as not to damage the ventricular wall or intracardiac structures such as papillary muscles, chordae tendinae, etc. The punch of the current invention allows for precise location and alignment of the cutting segment. The punch of the current invention also allows for precise advance of the cutting blade and a very clean cut of the tissue. Such clean cuts improve the healing when the hole in the body vessel or hollow organ is closed or attached to a connection, either prosthetic or natural.

### PRIORITY INFORMATION

This application is a continuation-in-part of U.S. patent application Ser. No. 09/938,428, filed Aug. 23, 2001, now U.S. Pat. No. 6,863,677.

### FIELD OF THE INVENTION

The field of this invention is related to instrumentation and devices for surgery and especially, interventional, cardiovascular, general or peripheral vascular surgery.

### BACKGROUND OF THE INVENTION

During surgical procedures such as placement of a ventricular assist device, blood vessel anastomosis, aortotomy, gastrotomy, enterotomy, or access to other hollow organs and vessels, it is useful to have a specialized tool to create a circular opening or fenestration in the wall of the vessel or organ. Punches have been developed for use in surgery that create such fenestrations. Examples of the prior art include U.S. Pat. No. 3,776,237 to Hill, U.S. Pat. No. 3,949,747 to Hevesy, U.S. Pat. No. 4,018,228 to Goosen, U.S. Pat. No. 4,122,855 to Tezel, U.S. Pat. No. 4,216,776 to Downie et al., U.S. Pat. No. 5,129,913 to Ruppert, U.S. Pat. No. 5,403,338 to Milo, U.S. Pat. No. 5,868,711 to Kramer et al., U.S. Pat. No. 5,827,316 to Young et al., U.S. Pat. No. 5,910,153 to Mayenberger, and U.S. Pat. No. 5,972,014 to Nevins. More recent patents include U.S. Pat. No. 6,033,419 to Hamblin, Jr. et al., U.S. Pat. No. 6,080,173 to Williamson IV et al., U.S. Pat. No. 6,080,176 to Young, U.S. Pat. No. 6,176,867 to Wright, and U.S. Pat. No. 6,187,022 to Alexander Jr. et al.

Problems with the current punches or coring devices occur both when the punch is positioned and actuated. With current systems, the cutting occurs by application of manual force by the surgeon. By requiring manual force to punch the hole in the organ or vessel wall without an adequate point of reference, the surgeon is not able to ascertain that the hole will be created along the correct path and at the selected location, prior to actually punching the hole. In addition, the current punches operate by means of a die without opposing back-up-plate cutting members. Examples of current punch mechanisms are similar to scissors where the cutting blade passes by an opposing brace or other cutting blade. These systems all create sub-optimal openings and leave ragged tissue edges.

New devices and methods are needed which facilitate creation of a hole in the hollow organ or vessel and allow confirmation of proper location, orientation, and coring path prior to actual creation of the hole in the hollow organ or vessel wall. In addition, devices are needed to make more precise, cleaner holes in the tissue. Such cleaner holes allow for more precise surgery, more controlled placement of anastomoses, more control over surgically created geometry, reduced blood loss and resultant improved patient outcome.

### SUMMARY OF THE INVENTION

This invention relates to a trephine, coring tool, or punch for creating a hole or stoma at a precise, desired location in a hollow organ or body vessel. The present invention is a cutting surface or edge that is opposed by an anvil to create a clean cut. The anvil comprises a tapered nose to facilitate penetration into the organ or vessel once a preliminary incision has been performed. The cutting surface or edge is spring loaded to perform the actual cutting under pre-assigned force. The system allows for location reference by allowing the punch to rest, under spring, or otherwise generated, force, against the tissue to be cut while final alignment is completed, thus allowing a more accurate cut. The system further provides for rotation of the cutting surface or edge as it approaches the anvil. Preferably, the cutting edge rotation is substantial, and greater than ¼ revolution (90 degrees) as it approaches, or is approached by, the anvil. The anvil in this type of system may be described as a Hammer Anvil since the face of the anvil that faces the cutting surface serves as a stop for the cutting surface as the distance between the anvil and the cutting surface or edge is reduced to zero.

In the prior art previously cited, including U.S. Pat. No. 4,018,228 to Goosen, U.S. Pat. No. 4,216,776 to Downie et al., U.S. Pat. No. 5,129,913 to Ruppert, U.S. Pat. No. 5,827,316 to Young et al., U.S. Pat. No. 5,910,153 to Mayenberger, U.S. Pat. No. 5,972,014 to Nevins, U.S. Pat. No. 6,080,173 to Williamson IV et al., and U.S. Pat. No. 6,080,176 to Young use a shearing or scissoring action between two blades to cut tissue. U.S. Pat. No. 3,949,855 to Hevesy, U.S. Pat. No. 4,122,855 to Tezel, and U.S. Pat. No. 6,187,022 to Alexander et al. use a knife or single sharpened edge with no opposing blade or surface to cut tissue. Both of these methods produce a ragged cut. The invention distinguishes over the cited prior art because the tissue is cut between a sharp edge and an opposing, flat, anvil-like surface to produce a clean cut. The embodiments of the punch disclosed herein provide further advantages over the prior art in that they create a hole that is closer to the diameter of the cutting edge than the holes made by the prior art punches.

The invention is most useful in cardiac surgery to create an opening or channel for cannula access to the ventricles of the heart or blood vessels near the heart. It is also useful for vascular surgery where side-to-side or end-to-side anastomoses need to be made. Alternatively, the system allows for general tissue biopsies and other general surgical applications on hollow organs or vessels such as a tracheostomy. Another aspect of the invention includes a method for creating a hole in a body vessel via an endovascular or interventional approach. Access to the vessel is created using a percutaneous approach such as the Seldinger technique. The method consists of creating an incision in the body vessel with a sharp object, inserting a sheath having a fluid-tight seal into the body vessel, and advancing a punch, further comprising a cutting blade and an anvil located at the distal end of a catheter, through the lumen of the sheath and extending out the distal end of the sheath into the body vessel until the distal end of the punch has reached a target location within the body vessel. The method further comprises advancing a sharp tip, affixed to the distal end of the punch, through the body vessel at the target site to create a puncture in the vessel wall. Next, an anvil with a tapered tip is advanced through the puncture in the body vessel at the target site and a circular cutting blade is located so that the cutting blade is positioned with the wall of the body vessel between the anvil and cutting blade. Next, the cutting blade is advanced through said body vessel wall under controlled force until the cutting blade fully rests against a distal surface of the anvil whose outside diameter is no less than the outer diameter of said cutting blade so that a hole is cut in the body vessel from the inside. The method further includes removing the cutting blade and excised tissue from the body vessel. It is advantageous that the cutting blade is rotated at least one revolution while said cutting blade is being advanced toward said anvil. It is further advantageous that a hemostatic plug or closure be provided to seal the vessel, generally on a temporary basis, immediately following creation of the punch hole and prior to further procedures on the vessel that require the presence of the punch hole.

For purposes of summarizing the invention, certain aspects, advantages and novel features of the invention are described herein. It is to be understood that not necessarily all such advantages may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein. These and other objects and advantages of the present invention will be more apparent from the following description taken in conjunction with the accompanying drawings.

### BRIEF DESCRIPTION OF THE DRAWINGS

A general architecture that implements the various features of the invention will now be described with reference to the drawings. The drawings and the associated descriptions are provided to illustrate embodiments of the invention and not to limit the scope of the invention. Throughout the drawings, reference numbers are re-used to indicate correspondence between referenced elements.

FIG. 1A illustrates a side view of the trephine, punch or coring tool with the cutter fully retracted, according to an embodiment of the invention;

FIG. 1B illustrates a side view of the trephine, punch or coring tool with the cutter fully advanced against the anvil, according to an embodiment of the invention;

FIG. 2 illustrates the trephine, punch or coring tool applied to the apex of the ventricle of the heart prior to advancing the cutting blade, according to an embodiment of the invention;

FIG. 3 illustrates the trephine, punch or coring tool after the blade has been advanced through the apex of the ventricular wall of the heart, according to an embodiment of the invention;

FIG. 4 illustrates the ventricular wall after removal of the trephine, punch or coring tool and the excised tissue, according to an embodiment of the invention;

FIG. 5A illustrates a side view of the trephine, punch or coring tool with the anvil fully advanced, according to an embodiment of the invention;

FIG. 5B illustrates a side view of the trephine, punch or coring tool with the anvil fully retracted against the cutter, according to an embodiment of the invention;

FIG. 6A illustrates a longitudinal cross-sectional view of the trephine, punch or coring tool comprising a jackscrew to replace the function of the spring, according to an embodiment of the invention;

FIG. 6B illustrates a side view of the trephine, punch or coring tool comprising the jackscrew, wherein the cutter has been advanced against the anvil, according to an embodiment of the invention;

FIG. 7A illustrates a side view of the trephine, punch or coring tool comprising a hydraulic cylinder to replace the function of the spring, according to an embodiment of the invention;

FIG. 7B illustrates a side view of the trephine, punch or coring tool comprising the hydraulic cylinder, wherein the cutter has been advanced against the anvil, according to an embodiment of the invention;

FIG. 8A illustrates a longitudinal cross-sectional view of the trephine, punch or coring tool comprising a jackscrew to replace the function of the spring, wherein the jackscrew is shown in detail, according to an embodiment of the invention;

FIG. 8B illustrates a side detailed view of the trephine, punch or coring tool comprising the jackscrew, wherein the cutter has been advanced against the anvil, according to an embodiment of the invention;

FIG. 9A illustrates a side detailed view of the trephine, punch or coring tool comprising a hydraulic cylinder to replace the function of the spring, according to an embodiment of the invention; and

FIG. 9B illustrates a side detailed view of the trephine, punch or coring tool comprising the hydraulic cylinder, wherein the cutter has been advanced against the anvil, according to an embodiment of the invention.

### DETAILED DESCRIPTION OF THE PREFERRED EMBODIMENTS

The invention may be embodied in other specific forms without departing from its spirit or essential characteristics. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

The invention, which is generally termed a surgical instrument, can be described as being an axially elongate structure having a proximal end and a distal end. The axially elongate structure further has a longitudinal axis. As is commonly used in the art of medical devices, the proximal end of the device is that end that is closest to the user, typically a surgeon. The distal end of the device is that end closest to the patient or that is first inserted into the patient. A direction being described as being proximal to a certain landmark will be closer to the surgeon, along the longitudinal axis, and further from the patient than the specified landmark.

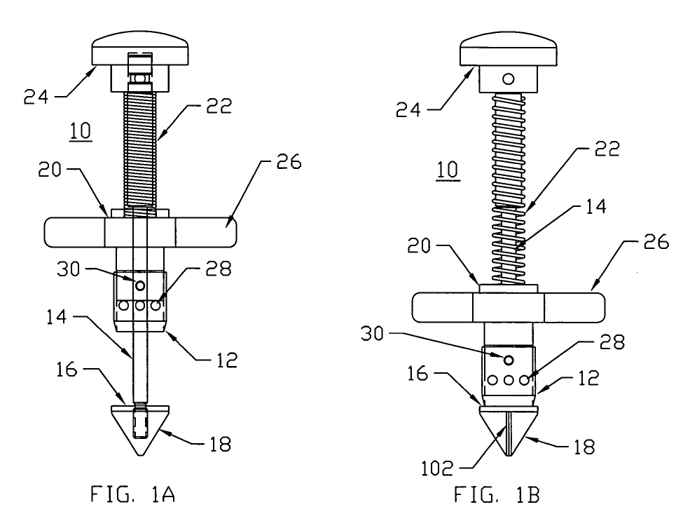

FIG. 1A illustrates a hollow organ coring tool, trephine, or punch 10 of the present invention. The coring tool 10 comprises a cutter 12, a central axially elongated shaft 14, an anvil 16, a trocar or tapered tip 18, a handle 20, a spring 22, and a knob 24. The cutter 12 comprises a plurality of holes 28. The handle 20 further comprises a plurality of wide flange-like members or wings 26. The handle 20 optionally comprises a setscrew 30. The cutter 12, the anvil 16, the trocar or tapered tip 18, the handle 20, the spring 22, and the knob 24 are disposed concentrically on the axially elongate shaft 14. The knob 24 is affixed to the proximal end of the shaft 14. The handle 20 is affixed to the cutter 12 with the optional setscrew 30. The handle 20 and attached cutter 12 slide rotationally and longitudinally in a one to one motion along and around the shaft 14. The spring 22 is slidably disposed between the knob 24 and the handle 20 and imparts a pre-determined force on the handle 20-cutter 12 assembly. The anvil 16 is affixed to the proximal end of the trocar or tip 18 and the tip 18 is affixed to the distal end of the shaft 14.

FIG. 1A shows the coring tool 10 with the cutter 12 in the fully retracted position. The cutter 12 is a cylindrical blade made from materials capable of being sharpened and with a high degree of hardness. Such materials include but are not limited to stainless steel, cobalt-nickel-chrome alloys, titanium alloys and the like. The cutter 12 has a sharpened configuration on its distal most edge to permit surgical cutting of body tissue. The distal cutting edge of the cutter 12 is, preferably, smooth but sharpened. Alternatively, the distal cutting edge may be serrated like a bread knife. The hollow interior of the cutter 12 is sufficiently long to allow the cored-out tissue to reside therein without being compressed. Holes 28 are optionally provided in the proximal end or sides of the cutter 12 to allow for fluid escape during cutting, thus preventing pressure buildup within the cutter 12. The cutter 12 may be any diameter necessary for the surgical procedure. The diameter of the cutter 12 ranges from 0.5 mm to 100 mm or even larger with the diameter range preferably being from 1 mm to 50 mm.

In another embodiment, the cutter 12 may be an electrocautery or electrocutting device consisting of an electrode. The electrode is electrically connected to a cable leading to one pole of an external electrocautery power supply. Another electrical pole of the power supply is an electrically conducting grounding pad electrically affixed to the patient's skin or other body organ, often with the aid of electrically conducting gel.

In a further embodiment, the cutter 12 may be rotationally vibrated using an electrical motor or one or more electrical actuators. Examples of electrical actuators include those fabricated from shape-memory nitinol with or without an elastic substrate. Ohmic heating of the nitinol actuators by application of electrical current causes reversible length change in said actuators. Opposably mounted actuators, energized one at a time, provide torque to rotationally vibrate the cutter 12 about the shaft 14. The actuators and cutter 12 operate at frequencies up to about 200 Hz. Electrical current is provided through an electrical cable leading to an external set of batteries and a controller. Alternatively, said controller and batteries could be mounted integral to the coring tool 10, such as in the knob 24, for example. Such rotational vibration makes the cutter 12 function like an electric bread knife with enhanced cutting capability over a stationary knife-edge. In another embodiment, however, a circumferential vibrational, reciprocating, or reciprocal motion using microactuators affixed at or near the distal end of the punch 10 can be performed. An electrical switch on the handle 20 or knob 24 (not shown) cause the microactuators to alternately pull the cutter 12 in one direction and then the other direction. The microactuators can be located at the distal end of the punch 10 and serve to vibrate or oscillate the cutter 12 circumferentially relative to the shaft 14. A description of the microactuators can be found in U.S. Pat. No. 5,405,337 to R. S. Maynard, the entirety of which is incorporated herein by reference. The vibrational motion generated by these microactuators is small and generally less than ¼ of a rotation. Further application of such actuators to cause rotational vibration of a device is disclosed in U.S. Pat. No. 6,110,121 to Lenker, the entirety of which is included herein by reference.

In a preferred embodiment, the handle 20 is affixed to the cutter 12. The handle 20 provides rotational force to the cutter 12 to assist in tissue penetration. The optional setscrew 30 may be used to attach the handle 20 to the cutter 12. Other ways to attach the handle 20 to the cutter 12 are the use of a rolled-pin, adhesives or over-molding. Mechanical advantage for manual rotation is derived from the wide flange like members or wings 26 on the handle 20 that allow increased moment arm to be applied to the handle 20 by the fingers of the surgeon. The handle 20 is preferably made from polymers such as but not limited to polycarbonate, acetal copolymers, acrylonitrile butadiene styrene, polyvinyl chloride and the like. The handle 20 optionally is provided with holes or openings that communicate with the optional holes 28 in the cutter 12 to allow for air and fluid escape from the interior of the cutter 12 through the handle 20 to the external environment during the coring process.

Optionally, the handle 20 comprises a latch or lock to maintain its position on shaft 14 in the retracted position under force of the spring 22. To move the handle 20 distally, the optional lock is released allowing the handle 20 to be advanced along the shaft 14 toward the anvil 16. The handle 20 further optionally comprises a damper or shock absorber to prevent the high velocity accidental release of the handle 20 and cutter 12 into the tissue.

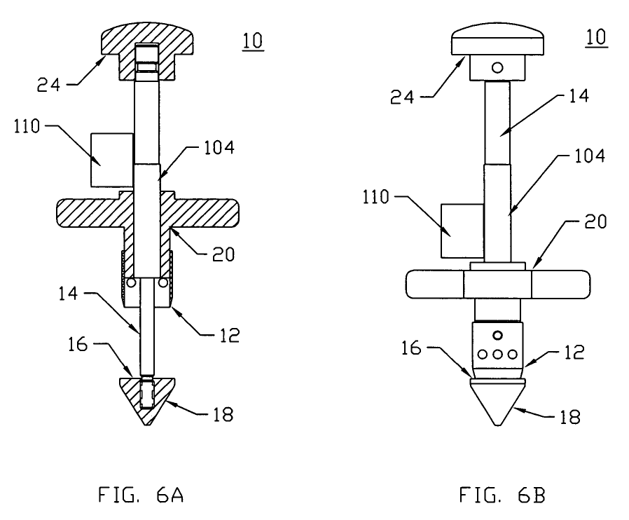

Alternatively, as illustrated in FIGS. 6A and 6B, the handle 20 may be rotated by a motor or gear motor 110, which is electrically powered by a battery disposed either external to or internal to the punch 10. External battery power is delivered to the motor 110 through a cable with a plurality of conductors. On and off operation of the motor 110 is controlled through a switch on the punch knob 24 or the handle 20, by a foot switch, or by a sound activated switch.

FIG. 1B shows the handle 20-cutter 12 assembly fully advanced against the anvil 16. The spring 22 is disposed between the knob 24 and the handle 20 and applies the desired force to the handle 20-cutter 12 assembly distally toward the anvil 16 with a pre-determined force. The pre-determined force is between 0.10 and 25 pounds and, preferably, between 1 and 10 pounds. This force is advantageous in performing a controlled tissue excision. The spring 22 also allows the cutter 12 to be disposed against the tissue prior to actual excision, without cutting, so that correct alignment may be determined by the surgeon. The spring 22 is, preferably, made from spring hardened metals such as stainless steel 304, stainless steel 316, nitinol, titanium alloys and the like. The spring 22 ensures that a seal is maintained between the cutter 12 and the tissue so that hemostasis is maximized or leakage of body fluids is minimized.

In another embodiment, as illustrated in FIGS. 6A and 6B, the function of the spring 22 is replaced by a threaded jackscrew assembly 104. The shaft 14 is threaded and engages mating threads on the handle 20. By rotating the handle 20, the cutter 12 is rotated and simultaneously advanced proximally or distally in a positive displacement fashion. FIG. 6A shows the coring tool 10 with the cutter 12 retracted away from the anvil 16. FIG. 6B shows the coring tool 10 with the cutter 12 advanced against the anvil 16. The anvil 16, serves as a stop for the cutter 12. This type of anvil 16 is also known as a hammer anvil.

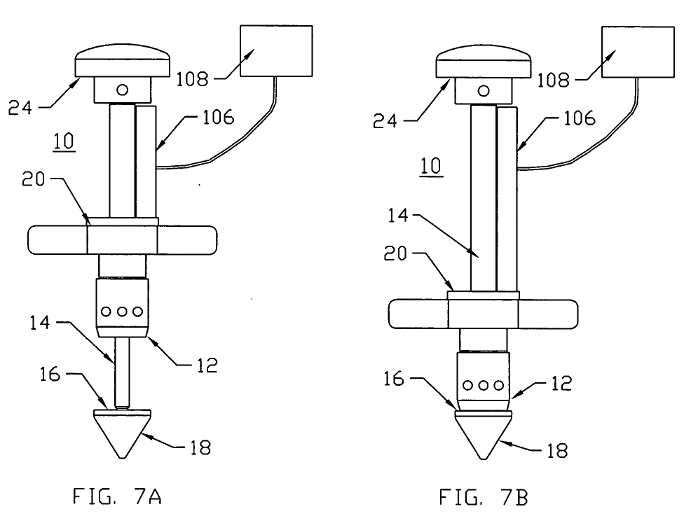

In yet another embodiment, as illustrated in FIGS. 7A and 7B, the function of the spring 22 is replaced by a hydraulic cylinder 106 and hydraulic pressure source 108 with a valve or switch to control pressure into said cylinder 106. FIG. 7A shows the coring tool 10 with the cutter 12 retracted away from the anvil 16. FIG. 7B shows the coring tool 10 with the cutter 12 advanced against the anvil 16.

The central shaft 14 maintains axial and longitudinal orientation of the punch 10 components. The shaft 14 is preferably fabricated from metals such as stainless steel, cobalt-nickel-chrome alloys, titanium alloys and the like. The shaft 14 may also be fabricated from hardened polymers such as glass-filled polycarbonate and the like. Holes or circumferential depressions in the shaft 14 permit attachment of components using setscrews or over-molding techniques. The shaft 14 geometry allows for expeditious replacement of optionally disposable components such as the cutter 12, anvil 16 and tip 18. The central shaft 14, optionally, comprises one or more circumferential alignment marks to confirm the position of the cutter 12 from the proximal end of the punch 10.

The tapered tip 18 is affixed to the distal end of the shaft 14 in a stationary manner. Fixation of the tip 18 to the shaft 14 is accomplished by over-molding, a setscrew or by internal threads on the trocar or tapered tip 18 engaging male threads on the shaft 14. The trocar or tapered tip 18 has a conical configuration and allows penetration of the hollow organ or vessel by the entire tip 18 anvil 16 assembly following an initial incision with a sharp surgical instrument. The distal end of the trocar 18 may be either sharp or rounded. Use of the sharp end on the trocar 18 permits use of the coring tool 10 without first making a separate surgical incision in the tissue. Longitudinal edges or ridges 102 are optionally disposed on the conical surface of trocar or tip 18 to enhance tissue penetration. Alternatively, the tip 18 may be oscillated or vibrated with an electrical actuator or motor to facilitate penetration into the tissue. The oscillation is useful for either blunt dissection or sharp dissection of the tissue.

The anvil 16 is a flat surface disposed distally to the cutter 12 and aligned in a plane generally perpendicular to the axis of the shaft 14. The anvil 16 is at least as wide as the largest exterior cutting dimension of the cutter 12. In this way, the anvil 16 serves to positively stop the cutter 12. The cutter 12 is advanced against the anvil 16 during the cutting procedure. The cutter 12 does not pass beyond the proximal surface of the anvil 16. In its lowest energy or inactive state, the cutter 12 rests against the anvil 16 with a net compressive force and the spring 22 expanded to its maximum allowable amount. The compressive force between the closed cutter 12 and the anvil 16 serves to maintain contact between the surfaces and promote cutting at the end of the stroke.

The anvil 16 and the tapered tip 18 are, preferably fabricated from the same piece of material for economy and ease of fabrication. Alternatively, the anvil 16 and the tapered tip 18 may be separate components and may be longitudinally disconnected or they may be longitudinally connected. Both the anvil 16 and the trocar or tapered tip 18 are radially constrained by the shaft 14. The anvil 16 is attached to shaft 14 by a setscrew, internal threads for engagement with male threads on the shaft 14, adhesive bonding or over-molding. The anvil 16 and the trocar or tapered tip 18 are, preferably, fabricated from polymeric materials such as but not limited to polyvinyl chloride, acetal copolymers, polycarbonate, acrylonitrile butadiene styrene and the like. They may alternatively be fabricated from metals such as stainless steel, cobalt-chrome-nickel alloys, titanium alloys and the like.

The anvil 16 optionally comprises pre-placed attachment devices, such as staples, sutures or posts that remain in the tissue around the coring site to facilitate subsequent placement of anastomotic devices.

The knob 24 terminates the proximal end of the shaft 14 and allows for positioning of the punch 10 by the surgeon. The knob 24 is blunt and preferably is fabricated from the same materials as the trocar or tip 18 or the anvil 16. The knob 24 is affixed to the shaft 14 with setscrews, adhesives, or over-molding or the knob 24 is affixed by female threads that engage male threads on the shaft 14.

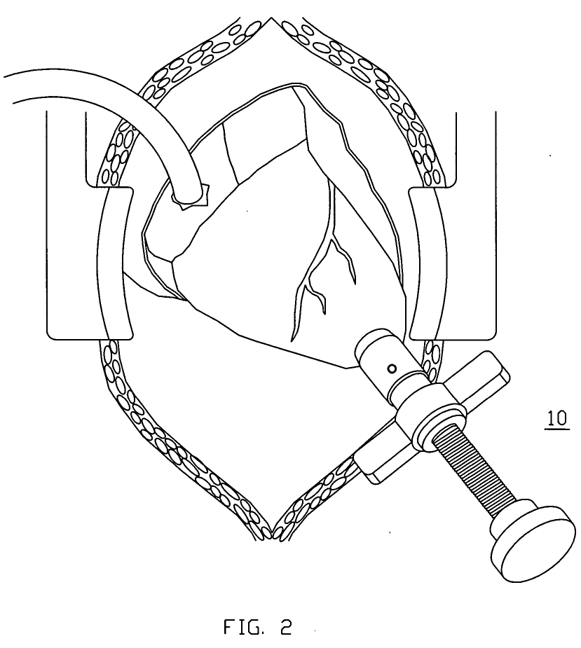

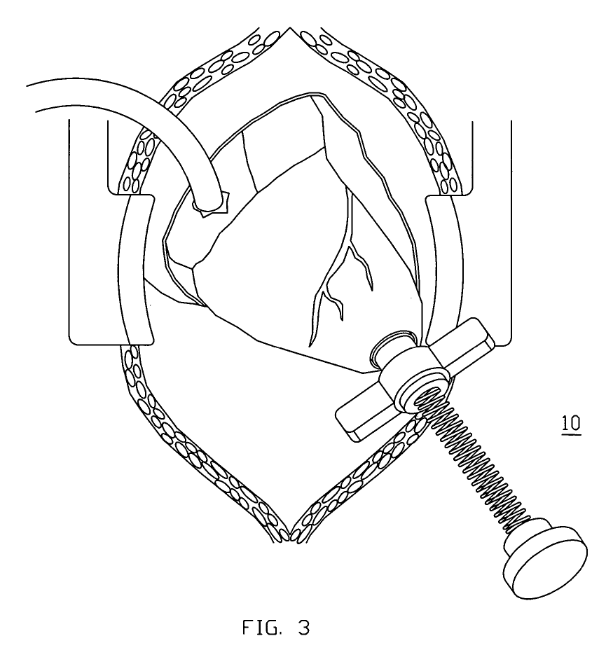

Referring to FIGS. 2, 3, and 4, the procedure for hollow organ coring or trephination is accomplished by first creating a small incision at the desired penetration location using a sharp surgical instrument such as a scalpel. The cutter 12 is retracted by manually withdrawing the handle 20 wings 26 proximally toward the knob 24. The spring 22 is compressed when retracting the handle 20 and cutter 12. The tapered tip 18 and anvil 16 assembly is advanced into the incision until the anvil 16 has passed beyond the interior surface of the hollow organ or vessel. The handle 20 is next released and the cutter 12 is positioned against the exterior of the hollow organ as shown in FIG. 2. Once position has been confirmed or adjusted, the handle 20 is manually rotated to initiate cutting of the tissue by the cylindrical cutter 12. As shown in FIG. 3, the handle 20 and cutter 12 are rotated until full penetration of the hollow organ has occurred, under force of the spring 22, and the distal edge of the cutter 12 rests against the anvil 16.

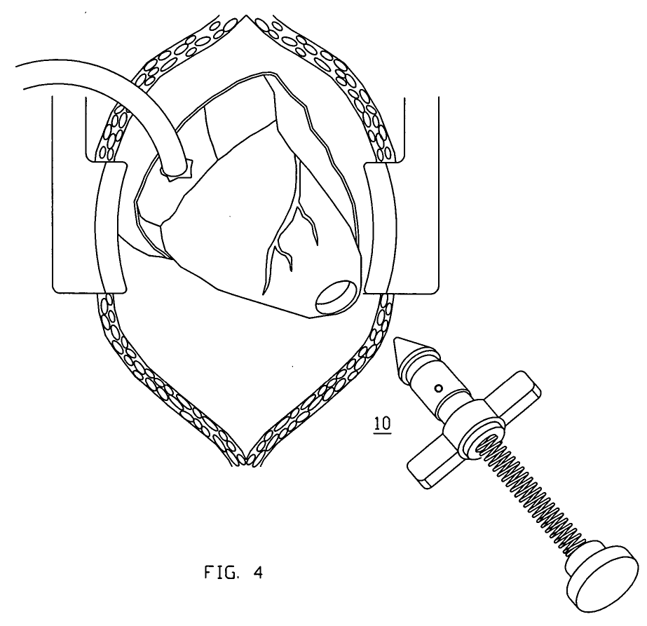

Complete penetration and cutter 12 to anvil 16 contact may be confirmed by placement of a plurality of alignment marks on the shaft 14. The alignment marks become visible once the cutter 12 and handle 20 have been advanced sufficiently. The punch 10 is next withdrawn proximally, removing the cored-out piece of tissue from the organ as shown in FIG. 4. Prevention of hemorrhage or fluid leakage from the hollow organ or vessel is accomplished by manual compression or placement of a temporary plug. This device and procedure are especially useful when performing coring on the beating heart.

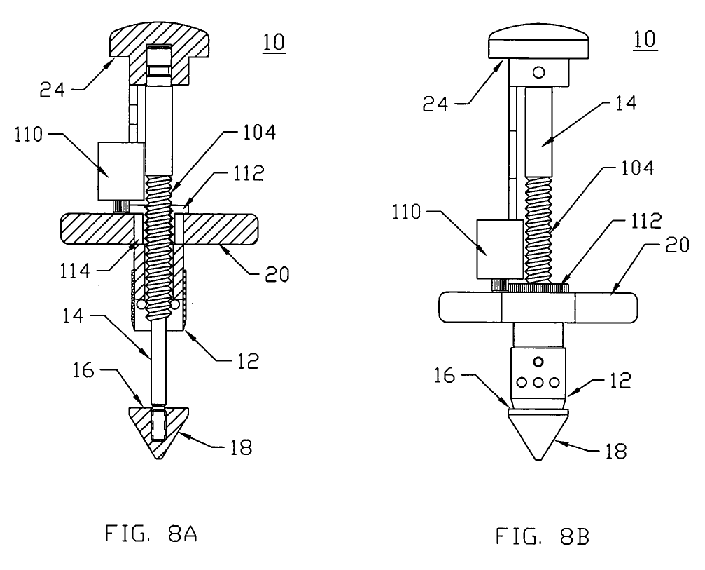

Typically, the surgeon manually cores the patient's hollow organ or vessel using the punch or coring tool 10. The coring tool 10 can alternatively, be held and manipulated by a robotic arm, endovascularly routed device such as a catheter, or a laparoscopic instrument. The laparoscopic instrument is generally placed through a sheath or trocar that has been inserted into the body through a percutaneous puncture site. In a laparoscopic embodiment, the shaft 14 is extended in length, relative to the device shown in FIG. 1A or FIG. 8A. The anvil 16 and tapered tip 18 reside at the distal end of the shaft 14 and are within the body distal to the distal end of the sheath. Furthermore, the region between the cutter 12 and the handle 20 is correspondingly extended in length so that the rotational force can be transmitted to the cutter 12, which resides within the body while the handle 20 and knob 24 are outside the body. Thus, all operational controls are outside the body and proximal to the proximal end of the laparoscopic sheath and a pressure seal. Visual control of the cutter is accomplished using a laparoscope routed through another trocar or sheath, or it is accomplished using ultrasound, fluoroscopy, or magnetic resonance imaging. The laparoscopic device is generally rigid and flexibility is not required, although it could be advantageous to make the shaft 14 flexible to allow some curvature. The laparoscopic device cutter and anvil 16 are generally between 1 and 15 mm in diameter. The length of the shaft 14 between the handle 20 and the proximal end of the cutter 12 can range between 5 and 50-cm.

An endovascular, interventional, or endoluminal device embodiment comprises a flexible shaft 14 that is capable of being routed through a sheath into a body vessel or lumen. The punch in this embodiment is affixed to a catheter. A hemostasis valve, fluid-tight seal or other gasket is provided at the proximal end of the sheath to prevent loss of blood, or body fluids, or the retrograde flow of air into the body. Typical cardiovascular access sheaths known in the art of endovascular access are appropriate for this application. The cutter 12 and anvil 16 reside at the distal end of the shaft 14. The shaft 14 is a torqueable axially elongate structure that also has column strength. The region between the handle 20 and the cutter 12 is generally very long in this embodiment. This length and the corresponding length of the shaft 14 may range from 10-cm to over 200-cm depending on the distance between the access site and the treatment site. The diameter of the cutter 12 is small enough to fit through the sheath, generally less than 24 French, or 8 mm in diameter. The cutter 12 and the anvil 16 can also be fabricated from structures that are radially expandable to allow them to fit through small diameter sheaths and then be enlarged to perform their coring function. The endovascular embodiment can also comprise a guidewire lumen (not shown) which is a central lumen extending from the proximal end of the knob 24 to the distal end of the tapered tip 18 so that the device can be routed over a guidewire, a slideable fit with a lumen diameter of 0.010 inches to 0.042 inches. All rotational operations and cutter 12 to anvil 16 closure operations are performed from the proximal end of the punch 10.

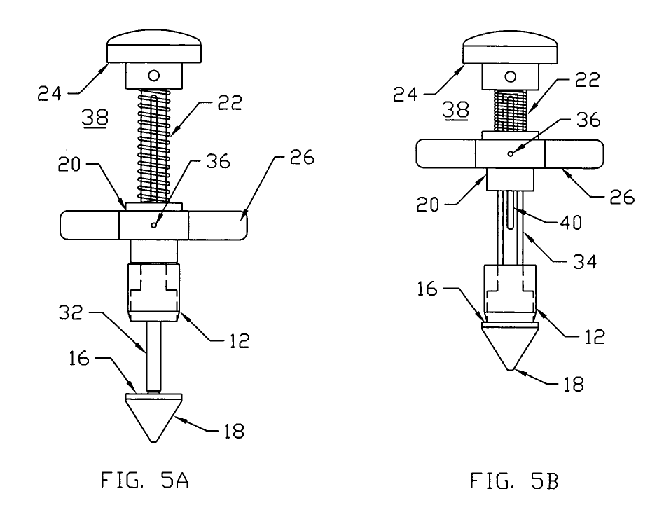

FIGS. 5A and 5B illustrate another embodiment of a hollow organ coring tool, trephine, or punch 38. The coring tool 38 comprises the cutter 12, the anvil 16, the trocar or tapered tip 18, the handle 20, the spring 22, and the knob 24. The coring tool 38 also comprises an inner shaft 32, an outer shaft 34, a pin 36, and an axial slot 40. The handle 20 further comprises the plurality of wide flange-like members or wings 26.

The cutter 12, the handle 20, the spring 22, and the knob 24 are disposed concentrically on the axially elongate outer shaft 34. The anvil 16 and the trocar or tip 18 are both disposed concentrically on the axially elongate inner shaft 32. The inner shaft 32 is slideably disposed inside the outer shaft 34 and the inner shaft 32 extends beyond the outer shaft 34 at least the thickness of the vessel or organ to be cored.

The handle 20 is not affixed to the cutter 12. Instead, the handle 20 is affixed to the inner shaft 32 by the pin 36 through the axial slot 40 in the outer shaft 34. The cutter 12 is affixed to the distal end of the outer shaft 34. The handle 20, which is affixed to the inner shaft 32, sets above the cutter 12, which is affixed to the outer shaft 34.

The knob 24 is affixed to the proximal end of the outer shaft 34. The anvil 16 is affixed to the proximal end of the trocar or tip 18 and the tip 18 is affixed to the distal end of the inner shaft 32.

The spring 22 sets around the outer shaft 34, between the knob 24 and the handle 20. The spring 22 forces the tip 18 and anvil 16 distally away from the cutter 12. Manual retraction of the handle 20 proximally causes proximal retraction of the anvil 16 toward the cutter 12. The spring 22 becomes increasingly compressed as the handle 20 is moved proximally toward the knob 24.

Referring to FIG. 5A, the handle 20, the tip 18, the anvil 16, and inner shaft 32 of the trephine 38 are fully advanced. The spring 22 is not compressed and is in its lowest energy position. The pin 36 rests in the distal end of the slot 40 and prevents the handle 20, the tip 18 and the anvil 16 from advancing further.

The handle 20 or the knob 24 optionally comprise a lock that is manually operated and selectively prevents movement of the inner shaft 32 relative to the outer shaft 34.

Referring to FIG. 5B, the handle 20, the tip 18, the anvil 16, and the inner shaft 32 are fully retracted. The spring 22 is fully compressed and in its highest energy position. Retraction of the handle 20 is accomplished with one hand over the knob 24 and fingers wrapped around the wings 26 in the handle 20. Pulling the fingers toward the knob 24 causes the anvil 16 to move proximally toward the cutter 12. The movement stops when the anvil 16 meets the cutter 12.

In an embodiment, as illustrated in FIGS. 8A and 8B, the function of the spring 22 of FIG. 1A is replaced by a threaded jackscrew assembly 104. The shaft 14 is threaded and engages mating threads on the handle 20. By application of an electrical energy source, such as a battery, which causes rotation of the motor 110, the cutter 12 is rotated. Optionally the cutter 12 may be simultaneously advanced proximally or distally in a positive displacement fashion by the threads 104 between the handle 20 and the shaft 14. This axial travel of the cutter 12 can be generated by the motor 110, rotation of the handle 20, or by a spring 22. The motor 110 can be a linear motor or it can be a rotational motor and actuate rotation of the cutter 12 through a gear assembly 112. A ratchet or rotational disconnect 114 can controllably and reversibly separate rotational motion of the handle 20 from the cutter 12. Actuation of the ratchet or rotational disconnect 114 can be accomplished through use of a lever or button (not shown) on the handle 20 or the knob 24. FIG. 8A shows the coring tool 10 with the cutter 12 retracted away from the anvil 16. FIG. 8B shows the coring tool 10 with the cutter 12 advanced against the anvil 16. The anvil 16, serves as a stop for the cutter 12. This type of anvil 16 is also known as a hammer anvil. FIGS. 8A and 8B illustrate a more detailed layout of the construction of one of the embodiments of the punch of FIGS. 6A and 6B.

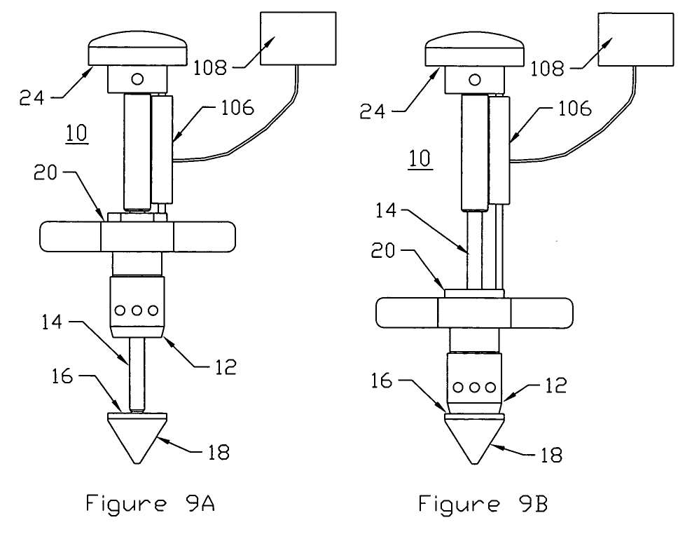

In an embodiment, as illustrated in FIGS. 9A and 9B, the function of the spring 22 is replaced by a hydraulic cylinder 106 and hydraulic pressure source 108 with a valve or switch to control pressure into said cylinder 106. FIG. 9A shows the coring tool 10 with the cutter 12 retracted away from the anvil 16. FIG. 9B shows the coring tool 10 with the cutter 12 advanced against the anvil 16. FIGS. 9A and 9B illustrate a more detailed layout of the construction of one of the embodiments of the punch of FIGS. 7A and 7B. In this embodiment, rotation of the cutter 12 is generated by rotating the handle 20. This cutter 12 rotation can also be generated by an electric motor or gear-motor 110 (see FIGS. 8A and 8B) or by a turbine (not shown) driven by the hydraulic pressure source 108.

In an embodiment, the cutter 12 is rotated by the motor 110. The cutter 12 is advanced toward the anvil 16, or the anvil retracted toward the cutter 12 by being biased by a spring 22. The cutter 12 can be retracted away from the anvil 16, or the anvil 16 advanced away from the cutter 12 by applying manual force to the handle 20 relative to the knob 24. It is beneficial that the cutter 12 be rotated substantially, in excess of ¼ revolution while approaching the anvil 16. Preferably, the cutter 12 is rotated in excess of 1 revolution as it approaches the anvil 16. Most preferably, the cutter 12 rotates two (2) or more times while approaching the anvil 16. This substantial rotation is beneficial in making the cleanest cuts in soft tissue. The substantial rotation is easily accomplished with a motor 110 or gear-motor. The substantial rotation can also be accomplished by manually turning the handle 20 or by a lever-ratchet assembly (not shown) with gearing to provide large rotational motion for a small amount of linear motion in the lever-ratchet assembly.

The procedure for hollow organ coring or trephination is accomplished by first creating a small incision at the desired penetration location using a sharp surgical instrument such as a scalpel. The tapered tip 18 and anvil 16 assembly is advanced into the incision until the anvil 16 has passed beyond the interior surface of the hollow organ or vessel and the cutter 12 rests on the exterior of the hollow organ or vessel. Once position has been confirmed or adjusted, the handle 20 is pulled toward the knob 24 to initiate cutting of the tissue by the circular cutter 12. The handle 20 is pulled until the distal edge of the cutter 12 rests against the anvil 16 and the organ has been cored. Complete penetration and cutter 12 to anvil 16 contact may be confirmed by placement of a plurality of alignment marks on the outer shaft 34. The alignment marks become visible once the anvil 16 and the handle 20 have been retracted sufficiently. The punch 38 is next withdrawn proximally, removing the cored-out piece of tissue from the organ.

The hollow organ coring tool, trephine, or punch 38 is fabricated from the same materials as the hollow organ coring tool, trephine, or punch 10 and comprises the same or similar options as the hollow organ coring tool, trephine, or punch 10. In an embodiment, the cutter 12 of the punch 38 can be rotated by a motor or actuator to facilitate tissue penetration.

The punch, in another embodiment, can comprise elements that plug or close the hole left behind following the coring procedure. The plug (not shown) can be a cylindrical or other axially elongate structure, affixed distal to the anvil such that it can be detached from the anvil 16. The plug is detached by actuation of a lever or other control element a the proximal end of the punch with the energy being mechanically, electrically, hydraulically, or pneumatically transmitted down the shaft 14 of the punch to the distal end, where a coupler is released to detach the plug. The plug can optionally comprise a line, tether, or string, routed out the proximal end of the punch, so that it can be removed from the tissue after a period of temporary placement. The punch, in another embodiment, can comprise suture elements that are routed through the tissue surrounding the punch hole. These suture elements, optionally tipped with needles or other sharp tissue penetration devices, can be captured and withdrawn from the proximal end of the punch to temporarily or permanently close the punch hole on itself or around a cannula or other axially elongate tube or vessel, placed therethrough. The needles or tissue penetration devices can be “J” shaped to permit easy recapture of the sharp distal end by mechanical motions generated within the punch. In yet another embodiment, the punch can comprise injection ports at its distal end for delivering adhesives to the punch hole site for the purposes of closure or enhanced anastomosis at the punch hole site. Adhesives, such as cyanoacrylate or other biological adhesives known in the art, can be stored in the shaft and injected by actuation at the proximal end, or they can be injected from the proximal end and delivered down the shaft 14 and exit at the injection ports at the distal end of the punch. Such adhesives can include single and multi-part adhesives that require mixing.

The present invention may be embodied in other specific forms without departing from its spirit or essential characteristics. The described embodiments are to be considered in all respects only as illustrative and not restrictive. The scope of the invention is therefore indicated by the appended claims rather than the foregoing description. All changes that come within the meaning and range of equivalency of the claims are to be embraced within their scope.

1. An apparatus adapted for cutting holes in a body vessel or hollow organ comprising:
a cutting blade,
a controlled force to advance the cutting blade, and
an anvil having a proximal surface against which the cutting blade is advanced,
wherein the cutting blade rotates at least ¼ turn relative to the anvil while the cutting blade is being advanced toward the anvil.

2. The apparatus of claim 1 wherein said controlled force on the cutting blade is generated by a spring with a pre-determined or selected spring constant.

3. The apparatus of claim 1 wherein the rotation of said cutting blade is generated by a lever assembly.

4. The apparatus of claim 1 wherein the rotation of the cutting blade is generated by a motor.

5. The apparatus of claim 1 wherein said rotation of the cutting blade is generated by manually turning a handle.

6. The apparatus of claim 1 wherein said cutting blade rotates at least two times while being closed against the anvil.

7. The apparatus of claim 1 wherein said cutting blade rotates at least one time while being advanced toward the anvil.

8. The apparatus of claim 1 wherein the cutting blade and anvil are located at the distal end of a long, flexible catheter while the handle and knob are located at the proximal end of the catheter.

9. The apparatus of claim 1 wherein said cutting blade and are advanced through a laparoscopic sheath.

10. The apparatus of claim 9 wherein said rotation of said cutting blade is generated by force applied proximally to the proximal end of said laparoscopic sheath.

11. The apparatus of claim 9 wherein said controlled force is generated proximally to the proximal end of said laparoscopic sheath.

12. The apparatus of claim 1 wherein said rotational force is generated by actuators that provide a reciprocating motion to the cutting blade.

13. A method for creating a hole in a body vessel comprising the steps of:
creating an incision in said body vessel with a sharp object,
inserting a sheath having a fluid-tight seal into said body vessel,
advancing a punch, further comprising a cutting blade and an anvil located at the distal end of a catheter, through said sheath into said body vessel until the distal end of the punch has reached a target location within the body vessel,
advancing a sharp tip, affixed to the punch, through the body vessel at the target site to create a puncture,
advancing an anvil with a tapered tip through the puncture in the body vessel at the target site,
locating a circular cutting blade so that said cutting blade is positioned with the wall of the body vessel between the anvil and cutting blade,
advancing said cutting blade into said body vessel wall under controlled force until said cutting blade fully rests against a distal surface of the anvil whose outside diameter is no less than the outer diameter of said cutting blade so that a hole is cut in the body vessel from the inside, and
removing said cutting blade and excised tissue from the body vessel,
wherein the cutting blade is rotated at least one revolution while said cutting blade is being advanced toward said anvil.

14. The method of claim 13 wherein the punch is introduced through a laparoscopic sheath rather than a vascular access sheath and wherein the hole is created in a hollow organ or body vessel from the outside, rather than the inside.

15. An apparatus adapted for cutting holes in a body vessel or hollow organ comprising:
an anvil,
a cutting blade against which the anvil is advanced wherein the anvil positively stops against the cutting blade, and
a controlled force to advance the anvil,
wherein the cutting blade rotates relative to the anvil at least ¼ turn while the anvil is being advanced toward the cutting blade.

16. The apparatus of claim 15 wherein said controlled force is generated by a spring to move the anvil against the cutting blade.

17. The apparatus of claim 15 wherein said rotation of the cutter is generated by a motor.

18. The apparatus of claim 15 wherein said rotation of the cutter is generated by an electrically powered microactuator that causes a reciprocal motion.

19. The apparatus of claim 15 wherein said rotation of the cutter is generated by a lever.

20. The apparatus of claim 15 wherein said rotation of the cutter is generated by manually turning a handle.
